(19) United States
(12) Patent Application Publication
(10) Pub. No.: US 2007/0292876 A1
(43) Pub. Date: Dec. 20, 2007

(54) METHODS FOR IDENTIFICATION OF COMPOUNDS THAT MIMIC THE SENESCENCE-INDUCING ACTIVITY OF RETINOIC ACID RECEPTOR LIGANDS

(75) Inventors: Yuhong Chen, Rochester, MN (US); Igor B. Roninson, Loudonville, NY (US)

Correspondence Address: KEOWN & ZUCCHERO, LLP, 500 WEST CUMMINGS PARK, SUITE 1200, WOBURN, MA, 01801, US

(73) Assignee: Ordway Research Institute, Inc.

(21) Appl. No.: 11/743,304

(22) Filed: May 2, 2007

Related U.S. Application Data
(60) Provisional application No. 60/746,195, filed on May 2, 2006.

(51) Int. Cl. C12Q 1/68

(52) U.S. Cl. 435/6

(57) ABSTRACT

The invention relates to the induction of senescence in neoplastic cells. More particularly, the invention relates to the use of small molecule compounds to mediate such induction of senescence. The invention provides methods for identifying such compounds, and methods for using the compounds.

This application claims the benefit of U.S. Provisional Application Ser. No. 60/746,195, filed on May 2, 2006. The entire teachings of the above-referenced applications are incorporated herein by reference.

### BACKGROUND OF THE INVENTION

1. Field of the Invention

The invention relates to the induction of senescence in neoplastic cells. More particularly, the invention relates to the use of small molecule compounds to mediate such induction.

2. Summary of the Related Art

Retinoids, natural and synthetic derivatives of vitamin A, are used in leukemia treatment and chemoprevention of cancers. These physiological regulators of gene expression were shown to be efficacious in the treatment of promyelocytic leukemia and (to a lesser degree) in chemoprevention of several cancers, in particular breast carcinoma.

Warrell, In Cancer, Principles and Practice of Oncology, V. T. H. S. DeVita and S. A. Rosenbert, eds. (Philadelphia: Lippincot Williams and Wilkins), pp. 489-494 (2001) teaches that retinoid treatment, however, produces a certain amount of systemic toxic responses, such as intracranial hypertension or hyperleukocytosis.

The antitumor effect of retinoids is most often attributed to the induction of differentiation (Altucci and Gronemeyer, Nat. Rev. Cancer 1, 181-193 (2001), but Roninson and Dokmanovic, J. Cell Biochem. 88: 83-94 (2003) teach that these compounds also stop the growth of tumor cells by activating the programs of apoptosis or senescence. Roninson and Dokmanovic, supra also teaches that (i) senescence is observed at the lowest and generally non-toxic concentrations of retinoids, and (ii) it involves upregulation of several growth-inhibitory proteins, including secreted factors that arrest the growth of neighboring non-senescent cells. Senescent tumor cells may therefore be regarded as a reservoir of secreted factors that provide for long-term inhibition of tumor growth.

Dokmanovic et al., PCT/US01/17161 teaches that retinoid-induced senescence of human MCF-7 breast carcinoma cells is associated with increased RNA expression of several intracellular and secreted proteins with known growth-inhibitory activities. These include actin-binding protein EPLIN (Epithelial Protein Lost in Neoplasm) and an ubiquitin-like protein UBD (formerly known as FAT 10), as well as secreted proteins insulin-like growth factor-binding protein 3 (IGFBP3) and an extracellular matrix component TGFBI (formerly known as βIG-h3). Induction of these genes can be used as the test for identifying other compounds that are likely to induce the same form of senescence as retinoids.

Induction of gene expression by retinoids is mediated at the level of transcription, through binding to dimeric transcription factors formed by retinoic acid receptors (RAR) and rexinoid receptors (RXR). The best-known mechanism of action of these retinoid receptors involves their binding to retinoic acid response elements (RARE) in the promoters of retinoid-responsive genes. Nevertheless, Altucci and Gronemeyer, supra teaches that retinoid receptors also affect transcription through RARE-independent mechanisms, such as repression of transcription factor AP-1 (Jun/Fos) and Husmann et al, Biochem. J. 352: 763-772 (2000) teaches that they can act or by modulating the interaction of Sp1 and GC-rich DNA via ternary complex formation. Retinoid receptors also interact with thyroid hormone receptor (Yen, Physiol. Rev. 81: 1097-1142, 2001), and retinoic acid can antagonize thyroid hormone action through plasma membrane interactions (Smith et al, Biochem J 284: 583-587, 1992).

Remarkably, Dokmanovic et al., Cancer Biology & Therapy 1:24-27 (2002) teaches that only one of 13 genes that were found to be strongly upregulated by retinoids in senescent MCF-7 cells, TRIM31, contains a putative RARE sequence in its promoter, whereas the other genes, including EPLIN, UBD, IGFBP3 and TGFBI, showed no identifiable RARE sequences. This suggests that retinoids upregulate these genes via a RARE-independent mechanism, but it is unknown whether this mechanism was mediated by retinoid receptors.

Induction of senescence in neoplastic cells is of special interest in anticancer drug development. There is, therefore, a need to develop compounds that can induce senescence in tumor cells.

### BRIEF SUMMARY OF THE INVENTION

In a first aspect, the invention provides methods for identifying a compound that induces senescence in mammalian cells. The method according to this aspect of the invention comprises providing mammalian cells, contacting the cells with a test compound, assaying the cells for the expression of at least one tumor-suppressor gene which is induced upon treatment by a retinoid and identifying the compound as capable of inducing senescence in mammalian cells if the tumor suppressor gene is induced in the presence of the compound.

In a second aspect, the invention provides methods for identifying a compound that induces senescence in mammalian cells. The method according to this aspect of the invention comprises providing mammalian cells, contacting the cells with a test compound, assaying the cells for the expression of at least one oncogene which is inhibited upon treatment by a retinoid and identifying the compound as capable of inducing senescence in mammalian cells if the oncogene is inhibited in the presence of the compound.

In a third aspect, the invention provides methods for identifying a compound that induces senescence in mammalian cells. The method according to this aspect of the invention comprises providing mammalian cells, contacting the cells with a test compound, assaying the cells for the expression of at least one tumor-suppressor gene which is induced upon treatment by a retinoid and for the expression of at least one oncogene which is inhibited upon treatment by a retinoid and identifying the compound as capable of inducing senescence in mammalian cells if the tumor-suppressor gene is induced and the oncogene is inhibited in the presence of the compound.

In a fourth aspect, the invention provides compounds identified by the first, second and third aspects of the invention, as well as optimized derivatives of such compounds.

### BRIEF DESCRIPTION OF THE DRAWINGS

FIG. 1 shows the effects of retinoid agonists and antagonists on MCF-7 cell growth. The bars represent cell number after 7 days of culture with the addition of DMSO (control), 100 nM RA, 100 nM RAR agonist LGD1550, 100 nM RXR agonist LGD1268, 10 μM RAR antagonist LG100815, and 10 μM RXR antagonist LG101208. Experiments were done in triplicate, and the results are expressed relative to the average of the control.

FIG. 2 shows the effects of retinoid agonists and antagonists on the senescent phenotype of MCF-7 cells. The bars represent percentages of SA-P-gal+cells after 8 days of treatment with the indicated compounds (in triplicate). The compounds were used at the same concentrations as in FIG. 1.

FIG. 3 depicts microarray analysis of changes in gene expression in MCF-7 cells treated with RAR ligands LGD1550 or LG100815, plotted using GeneSpring software. The X axis represents different time points of treatment with RAR ligands (0 point correspond to cells cultured for 3 days with DMSO carrier). The Y axis shows changes in gene expression on log scale. The groups of genes shown in panels (a) and (b) represent gene ontology (GO) categories, with the exclusion of genes showing raw signal intensity <10 in MCF-7 cells. The groups of genes shown in panels (c) and (d) represent the oncogenes and tumor suppressors listed in Table 1 and Table 2, respectively.

FIG. 4 shows the effects of coculture with all-trans retinoic acid (RA)-treated MCF-7 cells on MDA-MB-231 cell growth.
(a) MDA-MB-231 cells (GFP-expressing) were plated either alone or in 1:1 mixture with MCF-7 cells, in the presence of 100 nM RA or DMSO carrier. Each bar represents MDA-MB-231 cell number after five days of culture relative to cell number in the absence of MCF-7 or RA. Mean and standard deviation were calculated from three independent experiments.
(b) MDA-MB-231 cells (GFP-expressing) were plated as 1:1 mixtures with MCF-7 cells that were either untreated or treated for eight days with 100 nM, and grown in the presence of DMSO carrier or 100 nM RA. Each bar represents MDA-MB-231 cell relative to MDA-MB-231 cell number in coculture with untreated MCF-7 without RA. Mean and standard deviation were calculated from three independent experiments.

### DETAILED DESCRIPTION OF THE PREFERRED EMBODIMENTS

The invention relates to methods for identifying a compound that induces senescence in neoplastic cells. More particularly, the invention relates to the use of those compounds to mediate such induction of senescence. The patents and publications cited herein reflect the level of knowledge in the art and are hereby incorporated by reference in their entirety. Any conflict between the teachings of these patents and publications and this specification shall be resolved in favor of the latter.

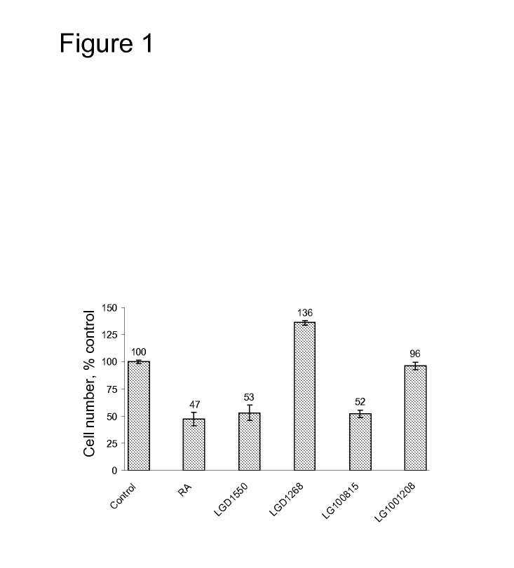

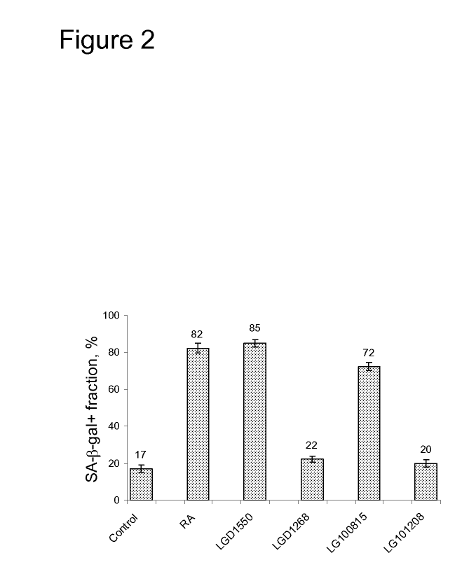

In the various aspects according to this invention, preferred compounds include retinoids, and more preferably ligands of retinoic acid receptors (RAR). As defined herein, a retinoic acid receptor ligand is intended to mean a retinoic acid receptor agonist (such as RA or LGD1550) and/or a retinoic acid receptor-modulating compound (e.g., a retinoic acid receptor antagonist such as LG100815), which are capable of inducing growth arrest and senescence in mammalian cells, as illustrated in FIG. 1 and FIG. 2.

In a first aspect, the invention provides methods for identifying a compound that induces senescence in mammalian cells. The method according to this aspect of the invention comprises providing mammalian cells, contacting the cells with a test compound, assaying the cells for the expression of at least one tumor-suppressor gene which is induced upon treatment by a retinoid and identifying the compound as capable of inducing senescence in mammalian cells if the tumor suppressor gene is induced in the presence of the compound. In preferred embodiments, the level of expression of at least one tumor-suppressor gene is normalized against cells not treated with the test compound. Suitable mammalian cells for use in the methods according to this aspect of the invention include, but are not limited to, neoplastic cells, hTERT-immortalized normal cells, or primary normal cell cultures.

In some embodiments the mammalian cells are proliferating cells such as, but not limited to, immortal cell lines. In some embodiments the proliferating cells are in a mammal, preferably the mammal is a human. As used herein, a tumor-suppressor gene includes, but is not limited to, those genes as listed in Table 2 below.

In certain embodiments of this aspect of the invention, induction of a tumor-suppressor gene can be determined by quantitative reverse-transcription PCR. This embodiment provides a method for identifying one or more compounds that induce senescence in mammalian cells by contacting the cells with a test compound, obtaining cells that have undergone senescence, determining the level of expression of at least one tumor-suppressor gene relative to cells not treated with the test compound.

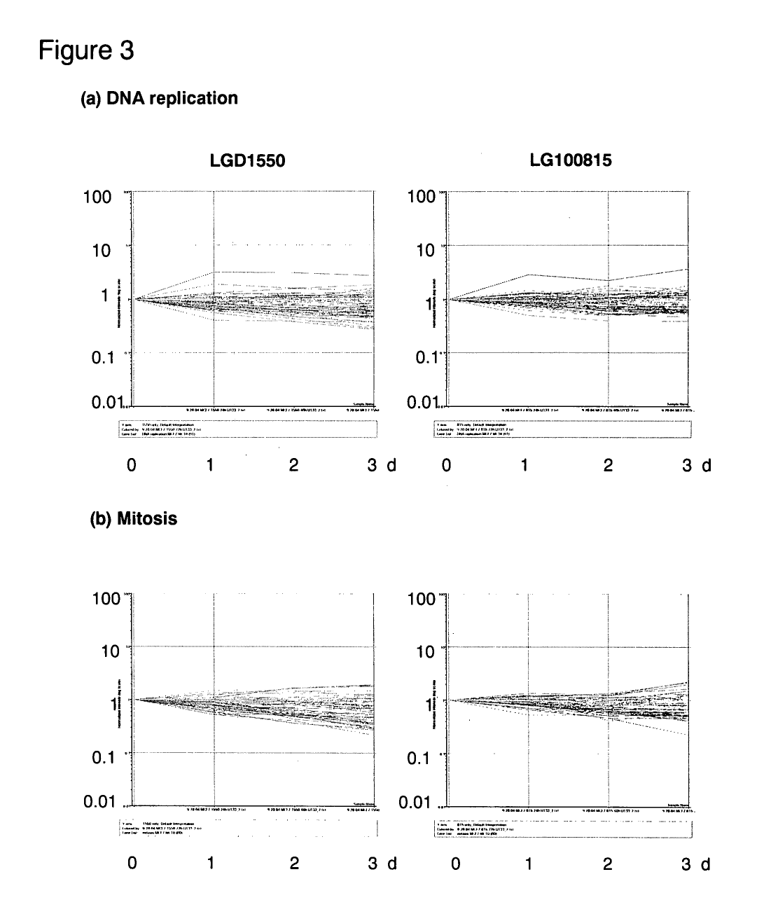

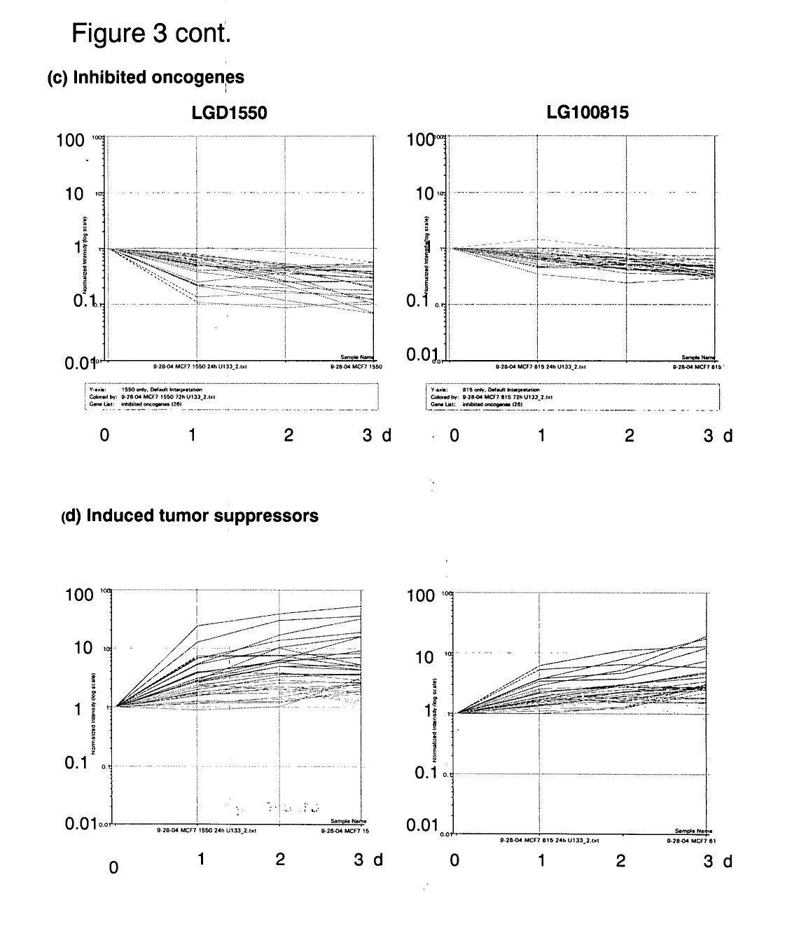

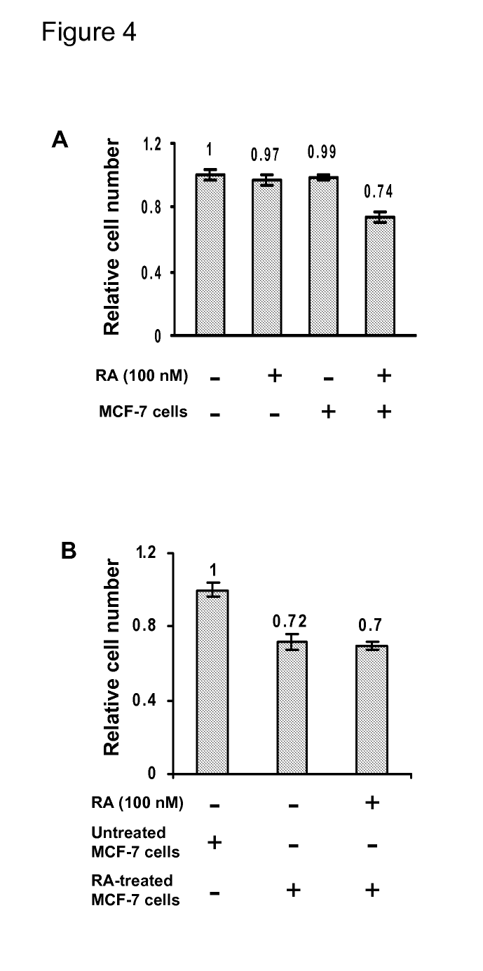

In certain embodiments of this aspect of the invention, induction of a tumor-suppressor gene can be determined by hybridization with oligonucleotide or cDNA arrays. For example, FIG. 3 depicts microarray analysis of changes in gene expression in MCF-7 cells.

In certain embodiments of this aspect of the invention, induction of a tumor-suppressor gene can be determined by providing cells transfected with a gene encoding a detectable protein operatively linked to a promoter of a tumor-suppressor gene, contacting the cells with a test compound, measuring the levels of detectable protein, and comparing the level of detectable protein expression relative to cells not treated with the test compound. Additionally, the expression of two or more tumor-suppressor genes can be simultaneously determined by providing cells further transfected with gene encoding a second detectable protein, that is different from and separately detectable in the presence of the first detectable protein, operatively linked to a promoter of a further tumor-suppressor gene. This embodiment provides a method for identifying one or more compounds that induce senescence in mammalian cells. Preferred detectable proteins include, without limitation, firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

In a second aspect, the invention provides methods for identifying a compound that induces senescence in mammalian cells. The method according to this aspect of the invention comprises providing mammalian cells, contacting the cells with a test compound, assaying the cells for the expression of at least one oncogene which is inhibited upon treatment by a retinoid and identifying the compound as capable of inducing senescence in mammalian cells if the oncogene is inhibited in the presence of the compound. In preferred embodiments, the level of expression of at least one oncogene is normalized against cells not treated with the test compound. Suitable mammalian cells for use in the methods according to this aspect of the invention include, but are not limited to, neoplastic cells, hTERT-immortalized normal cells, or primary normal cell cultures. In some embodiments the mammalian cells are proliferating cells such as, but not limited to, immortal cell lines. In preferred embodiments the proliferating cells are in a mammal, preferably the mammal is a human. The expression of an oncogene can be determined as described in the first aspect of the invention. As used herein, an oncogene includes, but is not limited to, those genes as listed in Table 1 below.

In a third aspect, the invention provides methods for identifying a compound that induces senescence in mammalian cells. The method according to this aspect of the invention comprises providing mammalian cells, contacting the cells with a test compound, assaying the cells for the expression of at least one tumor-suppressor gene which is induced upon treatment by a retinoid and for the expression of at least one oncogene which is inhibited upon treatment by a retinoid and identifying the compound as capable of inducing senescence in mammalian cells if the tumor-suppressor gene is induced and the oncogene is inhibited in the presence of the compound. In preferred embodiments, the level of expression of at least one tumor-suppressor gene and the expression of at least one oncogene is normalized against cells not treated with the test compound. Suitable mammalian cells for use in the methods according to this aspect of the invention include, but are not limited to, neoplastic cells, hTERT-immortalized normal cells, or primary normal cell cultures. In some embodiments the mammalian cells are proliferating cells such as, but not limited to, immortal cell lines. In preferred embodiments the proliferating cells are in a mammal, preferably the mammal is a human.

In certain embodiments of this aspect of the invention, induction of at least one tumor-suppressor gene and the inhibition of at least one oncogene can be determined by providing cells transfected with a first gene encoding a first detectable protein operatively linked to a promoter of a tumor-suppressor gene and a second gene encoding a second detectable protein, that is different from and separately detectable in the presence of the first detectable protein, operatively linked to a promoter of an oncogene. This embodiment provides a method for identifying one or more compounds that induce senescence in mammalian cells, by contacting the cells with a test compound, measuring the levels of first and second detectable proteins, and comparing the levels of detectable protein expression relative to cells not treated with the test compound. Test compounds that induce tumor-suppressor gene expression and inhibit oncogene expression are determined to be compounds that induce senescence in mammalian cells. Preferred detectable proteins include, without limitation, firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

Other methods for measuring the expression of a tumor-suppressor gene and an oncogene can be determined as described in the first and second aspects of the invention.

In a fourth aspect, the invention provides compounds identified by the first, second and third aspects of the invention, as well as optimized derivatives of such compounds.

The examples below are intended to further illustrate certain preferred embodiments of the invention, and are not intended to limit the scope of the invention.

### EXAMPLES

### Effects of RAR Ligands on Cell Growth and Senescence of MCF-7 Cells

MCF-7 cells were plated at 5×10⁵ per P100, and exposed on the next day to DMSO carrier, RA (100 nM), pan-RAR-specific ligands, LGD1550 (100 nM) and LG100815 (10 μM), pan-RXR specific agonist LGD1268, pan-RXR-specific antagonist LG101208, or drug-free DMSO carrier (control). The latter four compounds were provided by Ligand Pharmaceuticals, Inc. The effects of RAR ligands on the growth of MCF-7 cells were determined, as measured by the cell number after 7 days exposure to the compounds (FIG. 1) and on the fraction of cells expressing the senescence-associated β-galactosidase activity (SA-β-gal), a marker of senescence, after 7-day treatment with the compounds (FIG. 2).

The RAR-specific ligands LGD1550 and LG100815 inhibited the cell growth and induced SA-β-gal to an extent similar to that of RA, demonstrating that RAR stimulation is sufficient to induce senescence. The RXR agonist LGD1268 did not inhibit cell growth and did not induce the senescent phenotype; in fact, LGD1268 treatment produced a modest but reproducible increase in cell growth. The RXR antagonist LG101208 had no effect on the cell growth or the senescent phenotype.

Inhibition of Oncogenes by RAR Ligands

For the analysis of gene expression, cells treated with the carrier or with RAR ligands LGD1550 or LG100815 were collected after 24, 48 or 72 hr treatment. Total cellular RNA was isolated using Qiagen's RNeasy kit. For gene expression profiling, RNA samples were provided to the Microarray Core Facility at the Genomics Institute of the NYSDOH Wadsworth Center, which carried out biotinylated target preparation (using 2 μg RNA per assay) and hybridization with Affymetrix U133 Plus 2.0 microarrays. Data analysis was carried out using GeneSpring software (Agilent). Gene function analysis was carried out using Pathway Assist (Ariadne Genomics) and PubMed.

Gene Ontology groupings of genes with related biological functions (as provided in the GeneSpring software package) were surveyed for concerted response to RAR ligands. The most prominent biological categories of genes that are largely inhibited by both RAR ligands are genes involved in DNA replication (FIG. 3a) or mitosis (FIG. 3b). Strong downregulation of such genes has been associated with cell cycle arrest induced by chemotherapeutic drugs (Chang et al., 2002). In contrast, inhibition of these genes by RAR ligands was moderate (on average about 2-fold), with almost no genes showing 5-fold or stronger inhibition. On the other hand, we noticed that many of the genes that show the greatest fold inhibition by RAR ligands are known or putative oncogenes. Genes are defined herein as putative oncogenes or tumor suppressors if such genes have been reported in the literature to play a functional role in tumor cell growth, survival, tumorigenesis or metastasis, as determined by targeted inhibition, gene overexpression or protein addition studies. A total of 26 oncogenes were found to be inhibited by RAR ligands, with the strongest inhibition found for VAV3, SPDEF (a.k.a. PDEF), AMIGO2, MYB, RET, C4.4A and MAFB (Table 1). The time course of the inhibition of oncogenes listed in Table 1 is shown in FIG. 3c.

This surprising finding suggests that inhibition of oncogene mRNA expression may be an important component of retinoid-induced senescence. Novel compounds producing this desirable effect can be identified by their ability to inhibit the expression of these oncogenes or to suppress the activity of their promoters.

TABLE 1
Oncogenes inhibited by RAR ligands.

| | | | | Basal | Max fold | Max fold |
|---|---|---|---|---|---|---|
| | Affymetrix | | | raw | inhibition by | inhibition by |
| Gene Name | Probe No. | Genbank | Description | signal | LGD1550 | LG100815 |
| Cell-associated | | | | | | |
| VAV3 | 218807_at | NM_006113 | Vav 3 oncogene | 1181 | 14.5 | 2.7 |
| SPDEF | 220192_x_at | NM_012391 | SAM pointed domain containing ets | 522 | 14.2 | 3.0 |
| | | | transcription factor | | | |
| AMIGO2 | 222108_at | AC004010 | Adhesion molecule with Ig-like domain 2 | 1051 | 11.7 | 4.1 |
| MYB | 204798_at | NM_005375 | V-myb myeloblastosis viral oncogene | 716 | 9.9 | 2.2 |
| | | | homolog | | | |
| RET | 211421_s_at | M31213 | Ret proto-oncogene | 350 | 8.5 | 1.8 |
| C4.4A | 204952_at | NM_014400 | GPI-anchored metastasis-associated protein | 190 | 8.1 | 3.2 |
| | | | homolog | | | |
| MAFB | 218559_s_at | NM_005461 | V-maf musculoaponeurotic fibrosarcoma | 774 | 7.5 | 2.1 |
| | | | oncogene homolog B | | | |
| FGFR3 | 204379_s_at | NM_000142 | Fibroblast growth factor receptor 3 | 173 | 5.0 | 2.4 |
| GREB1 | 205862_at | NM_014668 | GREB1 protein | 285 | 4.8 | 2.8 |
| MSX2 | 210319_x_at | D89377 | Msh homeo box homolog 2 | 395 | 4.0 | 3.2 |
| PDLIM2 | 219165_at | NM_021630 | PDZ and LIM domain 2 (mystique) | 136 | 4.0 | 2.2 |
| ENTPD5 | 205757_at | NM_001249 | Ectonucleoside triphosphate | 51 | 3.4 | 3.0 |
| | | | diphosphohydrolase 5 | | | |
| MALAT1 | 224559_at | AF001540 | Metastasis associated lung adenocarcinoma | 200 | 2.5 | 3.4 |
| | | | transcript 1 | | | |
| PBX3 | 204082_at | NM_006195 | Pre-B-cell leukemia transcription factor 3 | 311 | 3.4 | 2.5 |
| CCNA2 | 203418_at | NM_001237 | Cyclin A2 | 415 | 3.3 | 2.3 |
| G6PD | 202275_at | NM_000402 | Glucose-6-phosphate dehydrogenase | 1380 | 2.2 | 3.2 |
| HMMR | 207165_at | NM_012485 | Hyaluronan-mediated motility receptor | 540 | 2.9 | 2.1 |
| | | | (RHAMM) | | | |
| CCND1 | 208711_s_at | BC000076 | Cyclin D1 | 695 | 2.1 | 2.9 |
| ECT2 | 219787_s_at | NM_018098 | Epithelial cell transforming sequence 2 | 545 | 2.8 | 1.6 |
| | | | oncogene | | | |
| PTTG1 | 203554_x_at | NM_004219 | Pituitary tumor-transforming 1 | 1236 | 2.7 | 2.2 |
| MYBL1 | 213906_at | AW592266 | V-myb myeloblastosis viral oncogene | 387 | 2.6 | 2.0 |
| | | | homolog-like 1 | | | |
| IRS1 | 204686_at | NM_005544 | Insulin receptor substrate 1 | 2117 | 2.6 | 1.6 |
| PIK3R1 | 212240_s_at | AI679268 | Phosphoinositide-3-kinase, regulatory | 1206 | 2.0 | 2.1 |
| | | | subunit 1 (p85 alpha) | | | |
| Secreted | | | | | | |
| CXCL12 | 209687_at | U19495 | Chemokine (C—X—C motif) ligand 12 (stromal | 596 | 5.7 | 1.6 |
| | | | cell-derived factor 1) | | | |
| TFF3 | 204623_at | NM_003226 | Trefoil factor 3 (intestinal) | 387 | 4.0 | 3.0 |
| BMP7 | 209591_s_at | M60316 | Bone morphogenetic protein 7 (osteogenic | 1008 | 1.8 | 2.0 |
| | | | protein 1) | | | |

Induction of Intracellular and Secreted Growth-Regulatory Proteins and Paracrine Growth-Inhibitory Activity of Retinoid-Treated MCF-7 Cells.

As previously described (Dokmanovic et al., 2002), retinoid treatment of MCF-7 cells induces genes that encode growth-inhibitory proteins, both intracellular (EPLIN and UBD) and secreted (IGFBP3 and TGFBI). Through the survey of the present microarray data, additional retinoid-inducible growth-inhibitory genes, induced both by LGD1550 and LG100815, have been identified. These genes, listed in Table 2, provide advantageous reporters for identifying compounds that mimic the senescence-inducing effects of retinoids. The products of these genes include both secreted factors and cell-associated proteins. The most highly induced cell-associated growth inhibitors were the previously identified UBD and EPLIN, followed by CEACAM 1, PPARG, MARCKS, BTG2 and NKX3-1. The most highly induced secreted tumor-suppressing proteins are TGFBI, IGFBP3, FBLN5 and GDF15. The time course of the induction of tumor suppressors listed in Table 2 is shown in FIG. 3d.

Since retinoid-treated MCF-7 cells upregulate genes for secreted factors with different activities, a functional test was carried out to determine whether retinoid-treated MCF-7 cells express primarily pro-mitogenic or anti-mitogenic activity. In this assay, MCF-7 cells were mixed (1:1) with MDA-MB-231 breast carcinoma cells (insensitive to retinoids), which were transduced with Green Fluorescent Protein (GFP), providing the ability to distinguish and quantitate these cells by FACS analysis. The co-cultures were treated for five days with 100 nM RA. As shown in FIG. 4a, RA or MCF-7 cells alone had no effect on the growth of MDA-MB-231 cells. In contrast, retinoid treatment in coculture with MCF-7 decreased the number of MDA-MB-231 cells by about 25% (FIG. 4a). In another type of experiment, shown in FIG. 4b, MCF-7 cells were pre-treated for 8 days with RA, to allow for complete growth arrest and development of the senescent phenotype. The treated MCF-7 cells were collected by trypsinization and cocultured for three days with MDA-MB-231, in the presence or in the absence of RA. Coculture with RA-pretreated MCF-7 cells was sufficient to inhibit MDA-MB-231 cell growth by about 30%, as compared to coculture with untreated MCF-7 cells (FIG. 4B, left bar). The addition of RA to the coculture had no significant effect on this growth inhibition (FIG. 4B, right bar), indicating that MDA-MB-231 cell growth was inhibited not by the retinoid but by factors secreted by retinoid-treated MCF-7 cells. These results indicate that MCF-7 cells rendered senescent by treatment with RAR ligands, produce the highly desirable tumor-suppressing activity.

RNA or protein products or promoter constructs of retinoid-inducible genes encoding tumor-suppressing intracellular or secreted proteins (Table 2) can be used as reporters in screening for compounds that mimic the senescence-inducing effect of retinoids. The most preferred reporters in this group include TGFBI (a.k.a. β-IG-h3), IGFBP3, UBD (a.k.a. FAT10) and EPLIN, previously identified as inducible in retinoid-induced senescence, as well as GDF15 (a.k.a. MIC-1 or PTGFβ) and BTG2 that were previously found to be upregulated in doxorubicin-induced senescence (Chang et al., 2002), and tumor-suppressive genes CEACAM1, PPARG, MARCKS, NKX3-1, and FBLN5, newly identified here as inducible by retinoids.

TABLE 2
Tumor suppressors induced by RAR ligands.

| | | | | | Max fold | |
|---|---|---|---|---|---|---|
| | | | | Basal | induction | Max fold |
| | Affymetrix | | | raw | by | induction by |
| Gene Name | Probe No. | Genbank | Description | signal | LGD1550 | LG100815 |
| Cell-associated | | | | | | |
| UBD | 205890_s_at | NM_006398 | Ubiquitin D | 36 | 35.9 | 17.5 |
| EPLIN | 217892_s_at | NM_016357 | Epithelial protein lost in neoplasm beta | 128 | 18.8 | 7.4 |
| CEACAM1 | 206576_s_at | NM_001712 | Carcinoembryonic antigen-related cell | 15 | 16.2 | 2.8 |
| | | | adhesion molecule 1 | | | |
| PPARG | 208510_s_at | NM_015869 | Peroxisome proliferative activated receptor, | 64 | 15.9 | 4.0 |
| | | | gamma | | | |
| MARCKS | 201669_s_at | NM_002356 | Myristoylated alanine-rich protein kinase C | 64 | 9.3 | 4.6 |
| | | | substrate | | | |
| BTG2 | 201236_s_at | NM_006763 | BTG family, member 2 | 87 | 8.3 | 6.5 |
| NKX3-1 | 209706_at | AF247704 | NK3 transcription factor related, locus 1 | 35 | 7.5 | 2.9 |
| IRF1 | 202531_at | NM_002198 | Interferon regulatory factor 1 | 43 | 5.3 | 2.4 |
| NBL1 | 37005_at | D28124 | Neuroblastoma, suppression of | 61 | 4.0 | 3.0 |
| | | | tumorigenicity 1 | | | |
| GADD45G | 204121_at | NM_006705 | Growth arrest and DNA-damage-inducible, | 37 | 3.8 | 2.7 |
| | | | gamma | | | |
| PHLDA1 | 217996_at | AA576961 | Pleckstrin homology-like domain, family A, | 359 | 3.7 | 2.7 |
| | | | member 1 | | | |
| HIPK2 | 225368_at | BF218115 | Homeodomain interacting protein kinase 2 | 114 | 3.7 | 2.8 |
| CDKN2B | 236313_at | AW444761 | Cyclin-dependent kinase inhibitor 2B (p15, | 14 | 3.2 | 3.1 |
| | | | inhibits CDK4) | | | |
| BATF | 205965_at | NM_006399 | Basic leucine zipper transcription factor, | 213 | 2.7 | 2.9 |
| | | | ATF-like | | | |
| VHL | 1559227_s_at | BF972755 | Von Hippel-Lindau tumor suppressor | 25 | 2.0 | 2.7 |
| EI24 | 216396_s_at | AF131850 | Etoposide induced 2.4 Mrna | 147 | 2.2 | 2.6 |
| KLF6 | 208961_s_at | AB017493 | Kruppel-like factor 6 | 25 | 2.2 | 2.5 |
| BTG1 | 200921_s_at | NM_001731 | B-cell translocation gene 1, anti-proliferative | 509 | 2.5 | 1.8 |
| DDIT3 | 209383_at | BC003637 | DNA-damage-inducible transcript 3 | 131 | 2.5 | 2.2 |
| FOXO3A | 224891_at | AV725666 | Forkhead box O3A | 1172 | 2.3 | 1.8 |
| GSN | 200696_s_at | NM_000177 | Gelsolin | 435 | 1.7 | 1.9 |
| PDCD4 | 212593_s_at | N92498 | Programmed cell death 4 (neoplastic | 1433 | 1.7 | 1.6 |
| | | | transformation inhibitor) | | | |
| Secreted | | | | | | |
| TGFBI | 201506_at | NM_000358 | Transforming growth factor, beta-induced, | 127 | 53.0 | 12.8 |
| | | | 68 kDa | | | |
| IGFBP3 | 210095_s_at | M31159 | Insulin-like growth factor binding protein 3 | 15 | 32.2 | 12.3 |
| FBLN5 | 203088_at | NM_006329 | Fibulin 5 | 7 | 10.1 | 19.0 |
| GDF15 | 221577_x_at | AF003934 | Growth differentiation factor 15 | 378 | 7.2 | 3.4 |
| SULF1 | 212354_at | BE500977 | Sulfatase 1 | 126 | 2.9 | 2.9 |
| TGFB2 | 228121_at | AU145950 | Transforming growth factor, beta 2 | 20 | 2.0 | 2.7 |
| IGFBP6 | 203851_at | NM_002178 | Insulin-like growth factor binding protein 6 | 81 | 2.6 | 2.1 |
| PRSS8 | 202525_at | NM_002773 | Protease, serine, 8 (prostasin) | 186 | 2.4 | 1.9 |
| PRSS11 | 201185_at | NM_002775 | Protease, serine, 11 (IGF binding) | 50 | 1.9 | 1.5 |

The novel finding that RAR ligands both increase the expression of multiple tumor suppressors and inhibit the expression of multiple oncogenes is of particular significance. This finding offers a particularly advantageous method of screening for compounds that mimic the senescence-inducing effect of retinoids. This method is based on measuring RNA or protein products or the activity of promoter constructs of both retinoid-inducible tumor-suppressors (Table 2) and retinoid-inhibited oncogenes (Table 1), to identify compounds that provide both the induction of at least one retinoid-inducible tumor suppressor and the inhibition of at least one retinoid-inhibited oncogene.

1. A method for identifying a compound that induces senescence in mammalian cells comprising providing mammalian cells, contacting the cells with a test compound, assaying the cells for the expression of at least one oncogene which is inhibited upon treatment by a retinoid and identifying the compound as capable of inducing senescence in mammalian cells if the oncogene is inhibited in the presence of the compound.

2. The method according to claim 1, wherein the oncogene is selected from the group consisting of VAV3, SPDEF, AMIGO2, MYB, RET, C4.4A, MAFB, FGFR3, GREB1, MSX2, PDLIM2, ENTPD5, MALAT1, PBX3, CCNA2, G6PD, CCND1, ECT2, PTTG1, MYBL1, IRS1, PIK3R1, CXCL12, TFF3 and BMP7.

3. The method according to claim 1, wherein the inhibition of at least one oncogene is determined by providing cells transfected with a gene encoding a detectable protein operatively linked to a promoter of an oncogene, measuring the levels of expression of the detectable protein, and comparing the level of detectable protein expression relative to cells not treated with the test compound.

4. The method according to claim 3, wherein the detectable protein is selected from the group consisting of firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

5. The method according to claim 1, wherein the inhibition of at least two oncogenes are determined by providing cells transfected with a first gene encoding a detectable protein operatively linked to a promoter of an oncogene and a second gene encoding a second detectable protein that is different from and separately detectable in the presence of the first detectable protein operatively linked to a promoter of a second oncogene, measuring the levels of expression of the detectable proteins, and comparing the levels of detectable protein expression relative to cells not treated with the test compound.

6. The method according to claim 5, wherein the detectable protein is selected from the group consisting of firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

7. The method according to claim 1, wherein the inhibition of the oncogene is determined by quantitative reverse transcription PCR.

8. The method according to claim 1, wherein the inhibition of the oncogene is determined by microarray analysis.

9. A method for identifying a compound that induces senescence in mammalian cells comprising providing mammalian cells, contacting the cells with a test compound, assaying the cells for the expression of at least one tumor-suppressor gene which is induced upon treatment by a retinoid and identifying the compound as capable of inducing senescence in mammalian cells if the tumor suppressor gene is induced in the presence of the compound.

10. The method according to claim 9, wherein the tumor-suppressor gene is selected from the group consisting of CEACAM1, PPARG, MARCKS, NKX3-1, IRF1, NBL1, GADD45G, PHLDA1, HIPK2, CDKN2B, BATF, VHL, E124, KLF6, DDIT3, FOXO3A, GSN, PDCD4, FBLN5, SULF1, TGFB2, PRSS8 and PRSS11.

11. The method according to claim 9, wherein the induction of a tumor-suppressor gene is determined by providing cells transfected with a gene encoding a detectable protein operatively linked to a promoter of a tumor-suppressor gene, measuring the level of expression of the detectable protein, and comparing the level of detectable protein expression relative to cells not treated with the test compound.

12. The method according to claim 11, wherein the detectable protein is selected from the group consisting of firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

13. The method according to claim 9, wherein the induction of at least two tumor-suppressor genes are determined by providing cells transfected with a first gene encoding a first detectable protein operatively linked to a promoter of a tumor-suppressor gene and a second gene encoding a second detectable protein that is different from and separately detectable in the presence of the first detectable protein operatively linked to a promoter of a second tumor-suppressor gene, measuring the levels of expression of the first and second detectable proteins, and comparing the levels of detectable protein expression relative to cells not treated with the test compound.

14. The method according to claim 13, wherein the detectable protein is selected from the group consisting of firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

15. The method according to claim 9, wherein the induction of the tumor suppressor gene is determined by quantitative reverse transcription PCR.

16. The method according to claim 9, wherein the induction of the tumor suppressor gene is determined by microarray analysis.

17. A method for identifying a compound that induces senescence in mammalian cells comprising providing mammalian cells, contacting the cells with a test compound, assaying the cells for the expression of at least one tumor-suppressor gene which is induced upon treatment by a retinoid and for at least one oncogene which is inhibited upon treatment by a retinoid; and identifying the compound as capable of inducing senescence in mammalian cells if the tumor-suppressor gene is induced and the oncogene is inhibited in the presence of the compound.

18. The method according to claim 17, wherein the tumor-suppressor gene is selected from the group consisting of UBD, EPLIN, CEACAM1, PPARG, MARCKS, BTG2, NKX3-1, IRF1, NBL1, GADD45G, PHLDA1, HIPK2, CDKN2B, BATF, VHL, E124, KLF6, BTG1, DDIT3, FOXO3A, GSN, PDCD4, TGFB1, IGFBP3, FBLN5, GDF15, SULF1, TGFB2, IGFBP6, PRSS8 and PRSS11.

19. The method according to claim 17, wherein the oncogene is selected from the group consisting of VAV3, SPDEF, AMIGO2, MYB, RET, C4.4A, MAFB, FGFR3, GREB1, MSX2, PDLIM2, ENTPD5, MALAT1, PBX3, CCNA2, G6PD, HMMR, CCND1, ECT2, PTTG1, MYBL1, IRS1, PIK3R1, CXCL12, TFF3 and BMP7.

20. The method according to claim 17, wherein the induction of at least one tumor-suppressor gene and the inhibition of at least one oncogene are determined by providing cells transfected with a first gene encoding a first detectable protein operatively linked to a promoter of a tumor-suppressor gene and a second gene encoding a second detectable protein that is different from and separately detectable in the presence of the first detectable protein operatively linked to a promoter of an oncogene, measuring the levels of expression of the first and second detectable proteins, and comparing the levels of detectable protein expression relative to cells not treated with the test compound.

21. The method according to claim 20, wherein the detectable protein is selected from the group consisting of firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

22. The method according to claim 17, wherein the induction of at least one tumor-suppressor gene and/or the inhibition of at least one oncogene are determined by quantitative reverse transcription PCR.

23. The method according to claim 17, wherein the induction of at least one tumor-suppressor gene and/or the inhibition of at least one oncogene are determined by microarray analysis.

24. A compound identified by the method of claim 1.

25. A compound identified by the method according to claim 9.

26. A compound identified by the method according to claim 17.
